(19) United States
(12) Patent Application Publication
(10) Pub. No.: US 2011/0201702 A1
(43) Pub. Date: Aug. 18, 2011

(54) ULTRA SMALL SYNTHETIC DOPED FERRIHYDRITE WITH NANOFLAKE MORPHOLOGY FOR SYNTHESIS OF ALTERNATIVE FUELS

(76) Inventors: Sumit Bali, Salt Lake City, UT (US); Garima Bali, Salt Lake City, UT (US); Edward M. Eyring, Salt Lake City, UT (US); Richard D. Ernst, Salt Lake City, UT (US); Ronald J. Pugmire, Salt Lake City, UT (US)

(21) Appl. No.: 12/857,387

(22) Filed: Aug. 16, 2010

Related U.S. Application Data
(60) Provisional application No. 61/234,146, filed on Aug. 14, 2009.
(60) Provisional application No. 61/309,763, filed on Mar. 2, 2010.

(51) Int. Cl. C07C 27/00; B01J 23/78; B01J 21/02; B01J 21/06; B01J 23/745; B01J 23/889; B01J 23/89; B01J 23/86; B01J 23/83; B01J 23/80; B01J 35/02; B01J 35/10; B01J 37/08; B01J 37/03

(52) U.S. Cl. 518/719; 502/330; 502/243; 502/331; 502/324; 502/326; 502/317; 502/303; 502/343

(57) ABSTRACT

A ferrihydrite catalyst composition can comprise a ferrihydrite of a structural promoter metal, a chemical promoter metal and potassium to form an amorphous nanoparticulate. The ferrihydrite catalyst can be formed by dissolving an iron salt, a structural promoter metal salt and a chemical promoter metal salt in water to form an aqueous iron solution. A ferrihydrite solid can be precipitated from the aqueous iron solution by addition of a precipitating agent under conditions such that the ferrihydrite solid is a nanoparticulate. A potassium can be incorporated into the ferrihydrite solid to form a ferrihydrite catalyst precursor. The ferrihydrite catalyst precursor can be calcined to form the ferrihydrite catalyst. A synthesis gas can be readily converted to a fuel product by contacting the ferrihydrite catalyst with the synthesis gas under reaction conditions sufficient to form a fuel product mixture.

### RELATED APPLICATIONS

This application claims the benefit of U.S. Provisional Patent Application No. 61/234,146, filed Aug. 14, 2009 and U.S. Provisional Patent Application No. 61/309,763, filed Mar. 2, 2010, and which are each incorporated herein by reference.

### GOVERNMENT INTEREST

This invention was made with government support under U.S. Department of Energy Grant No. DE-FC26-05NT42456. The United States government has certain rights to this invention.

### SUMMARY OF THE INVENTION

A ferrihydrite catalyst composition can comprise a structural promoter metal, a chemical promoter metal and potassium to form an amorphous nanoparticulate.

A method of forming this ferrihydrite nanoflake catalyst can involve dissolving an iron salt, a structural promoter metal salt and a chemical promoter metal salt in water to form an aqueous iron solution. A ferrihydrite solid can be precipitated from the aqueous iron solution by addition of a precipitating agent under conditions such that the ferrihydrite solid is a nanoparticulate. A potassium can be incorporated into the ferrihydrite solid to form a ferrihydrite catalyst precursor. This can be done susbsequent to or simultaneously with the precipitation. The ferrihydrite catalyst precursor can be calcined to form the ferrihydrite catalyst.

A method of converting a synthesis gas to a fuel product can include contacting the ferrihydrite catalyst with the synthesis gas under reaction conditions sufficient to form a fuel product mixture. Good CO conversion can be maintained even under reduced pressure and high flow conditions compared to conventional Fisher-Tropsch processes.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a graph of activity of UT-1-1.5 Ferrihydrite catalyst as a function of time on stream.

FIG. 2 is a flow chart of chemical products derived from synthesis gas.

FIG. 3 is a TEM image of a particle of Fe:Al:Cu:K (100Fe:25Al:5 Cu) by wt.

FIG. 4 is a schematic diagram of a fixed bed reactor system for F-T synthesis.

FIG. 5 shows catalyst evaluation in F-T reaction in a fixed bed reactor at 265° C. and 100 psi.

FIG. 6 is an XRD spectrum of a product mixture obtained using the catalysts in accordance with one aspect of the present invention.

FIG. 7 is a graph of activity versus time for two catalysts in accordance with aspects of the present invention.

FIG. 8 is a graph of activity versus time for catalysts supported on xerogel and aerogel in accordance with aspects of the present invention.

FIG. 9 is an XRD spectrum of as prepared Fe:Al:Cu ferrihydrite (UT-1) and potassium carbonate impregnated and calcined Fe:Al:Cu:K ferrihydrite (UT-2).

FIG. 10 is an XRD spectrum of reduced Fe:Al:Cu:K ferrihydrite (UT-3).

FIG. 11 is an XRD spectrum of spent (after F-T run) Fe:Al:Cu:K ferrihydrite (UT-11).

FIG. 12 is a Cu XANES spectra of UT-1 and UT-2 ferrihydrite.

FIG. 13 is an EMR spectra for the F-T catalyst UT-3 and spent catalyst UT-11 (after the F-T run).

FIG. 14A-14D are iron Mössbauer spectra of UT-1, UT-2, UT-3 and UT-11.

FIGS. 15A and 15B are TEM images of the synthesized Fe:Al:Cu (UT-1) and Fe:Al:Cu:K (UT-2) Ferrihydrite nanoflakes.

FIG. 16 is a graph of CO Conversion (g-CO/g-cat-h) using Fe:Al:Cu:K ferrihydrite nano flake catalyst.

FIG. 17 is a graph of quantitative carbon-13 NMR of F-T oil obtained using ferrihydrite nanoflake catalyst (Fe:Al:Cu 100:25:5 by weight; 1.5 wt % K₂CO₃).

FIG. 18 is a graph of DEPT NMR of F-T oil obtained using ferrihydrite nanoflake catalyst (Fe:Al:Cu 100:25:5 by weight; 1.5 wt % K₂CO₃).

FIG. 19 is a graph of quantitative carbon-13 NMR of F-T wax obtained using ferrihydrite nanoflake (Fe:Al:Cu:K) catalyst (Fe:Al:Cu 100:25:5 by weight; 1.5 wt % K₂CO₃).

FIG. 20 is a graph of DEPT NMR of F-T wax obtained using ferrihydrite nanoflake (Fe:Al:Cu:K) catalyst (Fe:Al:Cu 100:25:5 by weight; 1.5 wt % K₂CO₃).

FIG. 21 is a graph of CO Conversion (g-CO/g-cat-h) using Fe:Al:Cu:K ferrihydrite nanoflake catalyst with 0.75%, 1.5% and 3% K₂CO₃ by weight.

FIG. 22 is a graph of CO Conversion (g-CO/g-cat-h) using Fe:Al:Cu:K 100:25:10:1.5 ferrihydrite nanoflake catalyst with 1.5% K₂CO₃ by weight.

### DETAILED DESCRIPTION OF EXEMPLARY EMBODIMENTS

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### DEFINITIONS

In describing and claiming the present invention, the following terminology will be used.

The singular forms “a,” “an,” and “the” include plural referents unless the context clearly dictates otherwise. Thus, for example, reference to “a particle” includes reference to one or more of such materials and reference to “subjecting” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Ferrihydrite Catalysts with Promoter Metals

Ferrihydrites are iron oxide hydroxides which are nanoporous materials. Two types of materials are commonly called ferrihydrites. These are the amorphous 2-line ferrihydrites and 6-line ferrihydrites. This distinction is based on the number lines in their respective X-ray diffraction patterns. With high surface areas per unit volume, ferrihydrites are very reactive minerals. They can interact, either by surface adsorption or by co-precipitation. Owing to their high surface areas and pore volumes, the ferrihydrites are useful as catalysts and catalyst supports for a number of chemical transformations such as Fischer-Tropsch (F-T) synthesis, an important reaction for the conversion of syngas to alternative liquid fuels.

The Fisher-Tropsch (F-T) synthesis offers a way to convert synthesis gas which is a mixture of CO and H₂ obtained from coal, natural gas or biomass to a multicomponent mixture of gasoline, diesel fuel, waxes and other specialty chemicals.

(2n+1)H₂+nCO→CₙH₍₂ₙ₊₂₎+nH₂O F-T Synthesis (1)

Usual F-T catalysts used widely are derived from metals such as iron, cobalt and ruthenium. The use of iron catalysts is attractive because they are cheap and also have a high degree of water-gas shift (WGS) activity which is advantageous when using hydrogen-lean synthesis gas obtained from coal gasification.

CO+H₂O [symbol] CO₂+H₂ WGS reaction (2)

Syngas obtained from coal gasification typically has a H₂:CO ratio close to 1:1 whereas a higher proportion of hydrogen in syngas is clearly required by the above reaction stoichiometry.

An ferrihydrite catalyst composition can include a structural promoter metal, a chemical promoter metal and potassium to form an amorphous nanoparticulate. Each of these components contributes to the overall performance of the catalyst composition. The structural promoter metal can include at least one of Al and Si. In one aspect, the structural promoter metal is Al. The structural promoter not only appear to contribute to catalytic activity but is a factor in maintaining the amorphous structure of the catalyst composition. Similarly, the chemical promoter metal can include at least one of Cu, Mn, Pd, Ru, Cr, Pt, La, and Zn. The use of Cu as the chemical promoter metal can provide a good balance of performance and cost considerations. It can be beneficial to provide multiple chemical promoter metals within the composition. Combinations can include, but are not limited to, Cu—Mn, Cu—Pd, Cu—Zn, Mn—Zn, and the like. Non-limiting examples of catalyst composition combinations can include Fe/Al/Cu/K, Fe/Al/Cu/K, Fe/Al/Mn/K, Fe/Si/Mn/K, Fe/Cu/Mn/Al/K, Fe/Si/Cu/Mn/K, Fe/Al/Mn/Cu/K, Fe/Si/Mn/Cu/K, etc. In addition, precious metals or other secondary promoter metals can be incorporated into the catalyst composition, e.g. by gas phase incorporation, solution precipitation, or the like. Non-limiting examples of such precious metal containing catalyst compositions can include Fe/Cu/Al/Pd/K, Fe/Cu/Si/Pd/K, etc. Other additives such as structural binders can be added, for example, SiO₂, TiO₂, ZrO₂ and the like.

The catalyst composition also can be varied in terms of relative weight percentage of each component. Typically, the iron content dominates the composition and constitutes a majority of the composition. The structural promoter metal is generally the second most prominent component, followed by the chemical promoter metal, and potassium typically comprises the smallest percentage of the composition among these four constituents. As a general matter the catalyst composition can have an X:Y:Z ratio where X is the weight of Fe, Y is the weight of structural promoter metal and Z is the weight of chemical promoter metal. With Fe as a basis, X is 100. With this reference point and as a general guideline, Y is typically 20 to 35 and Z is 2 to 20, and in some cases Z is 2 to 10. In one specific example, the structural promoter metal is Al, the chemical promoter metal is Cu, and the composition has a Fe:Al:Cu ratio of about 100:25:5 by weight. This appears to be a nearly optimal ratio of components for this combination of components in F-T reaction conditions.

Similarly, the potassium content can affect catalytic performance of the composition. Excess potassium can tend to deactivate the catalyst composition while insufficient potassium can substantially reduce the amount of olefins produced. As a general guideline, the potassium can be present at about 0.7 to about 3.0 weight percent of the composition. In one specific aspect, the potassium is present at about 1.5 weight percent of the composition.

The catalyst compositions also benefit from having an extremely small particle size. The particle morphology appears to be an amorphous nanoflake material. Regardless, the catalyst is a nanoparticulate. Generally, the nanoparticulate has an average size of about 5 nm to about 20 nm. The properties of these nanoflakes are unique. For example, a blocking temperature (T_{B}) of ˜20 K and spin-glass transition temperature (T_{S}) of 6 K, and lower magnitudes of average magnetic moment (μₚ) of 70 μ_{B} per flake were calculated from the data. These lower magnitudes can be explained in terms of the smaller effective volume of the nanoflakes by a factor of about ⅓ as compared to the volume of known 5 nm spherical FHYD particles. The weaker and broad EMR line observed in this system is consistent with the structural disorder produced by doping with Al and Cu. The unique morphology of this sample combined with its smaller effective volume is a source of excellent catalytic properties. In one aspect, the nanoparticulate can have a blocking temperature from about 10 K to about 30 K and a spin-glass transition temperature of less than about 15 K. Although other surface areas can be achieved, typically, the nanoparticulate has a BET surface area prior to potassium loading from about 310 m²/g to about 380 m²/g, and often from about 315 m²/g to about 340 m²/g.

A method of forming the ferrihydrite catalyst can include dissolving an iron salt, a structural promoter metal salt and a chemical promoter metal salt in water to form an aqueous iron solution. The salts are dissolved to provide the corresponding metal. Although other salts can be used, nitrate salts, sulfate salts, and hydrated chlorides of Fe(III) are suitable. Specific examples of suitable salts can include, but are not limited to, used to iron nitrate, iron sulfate, aluminum nitrate, aluminum sulfate, copper nitrate, copper sulfate, manganese nitrate, manganese sulfate, palladium nitrate, palladium sulfate, zinc nitrate, and zinc sulfate. Incorporating multiple structural and/or chemical promoter metals can involve dissolving combinations of these salts, although such additional metals can optionally be incorporated in subsequent steps. Most often, no additional heating is required to dissolve these salts, such that room temperature mixing is sufficient. However, in some cases, the salts can be heated slightly to increase dissolution (e.g. 20° C. to about 40° C.

Once the aqueous iron solution is formed, the metals can be precipitated to form a nanoparticulate ferrihydrite solid. This can be accomplished using a precipitating agent such as a basic solution, although other agents can be used. For example, drying (i.e. solvent removal) can allow for precipitation. Non-limiting examples of suitable precipitating agents include Na₂CO₃, KOH, NaOH, NH₄HCO₃, mixtures thereof (e.g. Na₂CO₃:NaOH) or the like. Advantageously, the precipitation conditions can include a low temperature from about 20° C. to about 35° C. Excess heat can cause agglomeration of particles, thus increasing particle size and decreasing performance. Currently, a precipitation temperature of about 25° C. provides suitable results. Typically, the concentration of the metals in solution corresponds to the desired concentration in the final precipitated solids. For example, the precipitating agent added normally completely precipitates the metals out. Based on the moles of each present, the required amount of base for each would be used, e.g for 1 mole of Fe³⁺ 1.5 moles of Na₂CO₃; for 1 mol Al³⁺ 1.5 moles of Na₂CO₃; and for 1 mole Cu²⁺ 1 mole Na₂CO₃.

The potassium can be incorporated into the ferrihydrite solid to form an ferrihydrite catalyst precursor. This can be accomplished in any suitable manner. For example, the potassium can be impregnated into the precipitated ferrihydrite solid to form a ferrihydrite catalyst precursor. Wet impregnation can be performed by exposing the solid to a potassium solution and then evaporating the water to leave the potassium salt deposited onto the ferrihydrite solid. Alternatively, K₂O can be physically mixed with the ferrihydrite precursor.

The ferrihydrite catalyst precursor can then be calcined to form the ferrihydrite catalyst. Calcining can help to remove residual moisture and convert the metals to their respective oxides.

The ferrihydrite catalyst can be incorporated onto a support material. This can be accomplished using any suitable method such as, but not limited to, wet impregnation, gas phase incorporation, supercritical drying, or air drying. The support material can be any suitable support material such as aerogel, xerogel, ceramic (e.g. alumina, silica, zeolites, etc.) and the like. These supports can be structured (e.g. honeycomb, pelletized, etc) or particulate. In one aspect, the support material is at least one of an aerogel and a xerogel.

The above-described ferrihydrite catalysts can be particularly useful in converting synthesis gas into fuels or other useful compounds. Fisher-Tropsch reaction pathways are of particular interest due to the availability of renewable synthesis gas sources and variety of fuel products which can be derived. FIG. 2 illustrates some of the many products which can be formed from synthesis gas (i.e. mixture of CO and H₂). The F-T process allows conversion of synthesis gas to products such as gasoline, diesel, kerosene, waxes, naptha and the like. Variations in specific catalysts and conditions can increase or decrease the fraction of different products such as olefins, saturated hydrocarbons, etc.

A method of converting a synthesis gas to a fuel product can include contacting the ferrihydrite catalyst with the synthesis gas under reaction conditions sufficient to form a fuel product mixture. The reaction can be carried out in any suitable reactor such as a fixed bed reactor or a slurry reactor, although other reactors can be used.

Specific reaction conditions can also vary. However, the ferrihydrite catalysts can allow for relatively lower pressures (especially in fixed bed reactors) while still maintaining good CO conversion. In one aspect, the reaction conditions can include a pressure from about 75 psi to about 150 psi. Similarly, in another aspect, the reaction conditions can include a temperature from about 200° C. to about 280° C. Flow rates can be varied, although good CO conversion can be achieved when the reaction conditions include a H₂ space velocity from about 1.068 hr⁻¹ to about 2.136 h⁻¹. Similarly, the reaction conditions can include a CO space velocity from about 7.5 hr⁻¹ to about 15 hr⁻¹. Although actual CO conversion can vary, these conditions can often lead to CO conversion of above 40% over 100 hour on stream conditions.

Optionally, the ferrihydrite catalyst can be used in combination with other catalysts. For example, the reaction can include simultaneously contacting the synthesis gas with a zeolite catalyst (e.g. ZSM-5) and the ferrihydrite catalyst. This can lead to increase in gasoline and diesel fractions via cracking and aromatization of long-chain paraffins. Furthermore, the ferrihydrite catalysts can exhibit good stability over time in F-T processing. Typically, the ferrihydrite catalyst can be maintained under the process conditions for a reaction time on stream of about 70 hours to about 120 hours with less than 2% loss in CO conversion activity.

The resulting fuel products can be fractionated, further processed, and/or used as formed. One additional advantage of the ferrihydrite catalysts is the production of very few oxygenates (i.e. alcohols, ethers, etc.). As a general rule, the fuel product includes less than about 0.7 wt % oxygenates, and in some cases less than about 0.1 wt %.

### EXAMPLES

### Example 1

Iron based ferrihydrite type catalysts for F-T synthesis were synthesized. Transmission electron microscopy carried out on synthetic ferrihydrites showed nano-flakes of about 5-20 nm size without any hint of diffraction fringes that are characteristic of crystalline nature. Hence these nanoflakes did not exhibit any noticeable crystalline order and had an effective magnetic size of 2.5 nm. These ultrasmall synthetic ferrihydrite nanoflakes exhibit unique properties and have been characterized extensively by magnetic measurements, X-ray diffraction, electron microscopy as well as by Mössbauer spectroscopic studies. The synthesis and magnetic properties of a synthetic 2-line FHYD showed a considerably lower blocking temperature (T_{B}=20K), spin-glass transition T_{g}=6 K and magnetic size=2.5 nm. This sample prepared for catalysis applications in Fischer-Tropsch synthesis contained the metallic ratios of Fe:Al:Cu=100:25:5 by weight. In spite of the presence of Al and Cu usually absent in natural FHYD, the 2-line FHYD structure was confirmed by XRD and FTIR spectroscopy. Analysis of the magnetization M vs. applied field H up to 65 kOe and temperature T (2K to 300K) yielded the above quoted magnitude of T_{B}, T_{g} and D.

Although T_{g}≈6 K is indicated in the M vs. T data by a weak anomaly, its presence was confirmed by meaning the coercivity H_{c} in a zero-field cooled (2FC) and FC (field cooled) sample in 20 kOe. For T<T_{g}, exchange-bias H_{eb} appears and H_{c}(FC)>H_{c}(ZFC). Magnitude of the magnetic D≈2.5 nm was determined by analyzing the M vs. H data in the low field and the high field regions. Synthesis of this ultra small ferrhydrite (FHYD) with D≈2.5 nm represents a new way to produce FHYD nanoparticles with ultra small dimensions not usually found in nature.

Synthesis of Ferrihydrite UT-1 (Fe:Al:Cu 100:25:5 by Weight)

Hydrated nitrates of iron (Fe(NO₃)₃.6H₂O, 36.1813 g), Aluminum (Al(NO₃)₃.9H₂O, 17.3656) and copper (Cu(NO₃)₂.2.5H₂O, 0.9188 g) were dissolved in pure (Mili-Q) water in a 200 mL standard volumetric flask to give solution A. Sodium carbonate ((Na₂CO₃, 22.0180 g) was dissolved in pure (mili-Q) water in 200 mL standard volumetric flask to give solution B. Both solution A and solution B were then added drop wise simultaneously through burettes into a 600 mL beaker with vigorous stirring. An orange colored precipitate started forming. After complete addition of both the solutions, the precipitate was aged (with stirring) for another 24 hrs at room temperature. The orange colored precipitate (UT-1) was filtered and washed with large quantities of water. The precipitate was dried overnight in air in an oven at 100° C.

Wet Impregnation of Potassium Carbonate (K₂CO₃) onto UT-1: Synthesis of UT-1-1.5

A weighed amount of crushed UT-1 was taken in a glass vial to which solution of potassium carbonate (1.5 wt % of UT-1) in water was added with stirring. The solution was added in such a manner so as to completely submerge the entire UT-1 precipitate. The vial was then kept in an oven in air overnight at 110° C. to remove the water and hence impregnate K₂CO₃ on UT-1. The dried Fe Cu Al K catalyst (UT-1-1.5) was stored under ambient conditions

Thermal Processing (Calcination) of UT-1-1.5

UT-1-1.5 was calcined under flowing air (200 mL/min) in a furnace with heating at a rate of 10° C./min, holding at 400° C. for 4 h and finally cooling it back to room temperature.

Reduction of UT-1-1.5 Catalyst

Prior to loading in the reactor for evaluation of F-T activity, the synthesized UT-1-1.5 catalyst was reduced under flowing H₂ 50 ml/min with heating at 10° C./min up to 280° C. and holding it at 280° C. for 8 h and finally cooling it to room temperature under H₂

Catalyst Evaluation for F-T Activity.

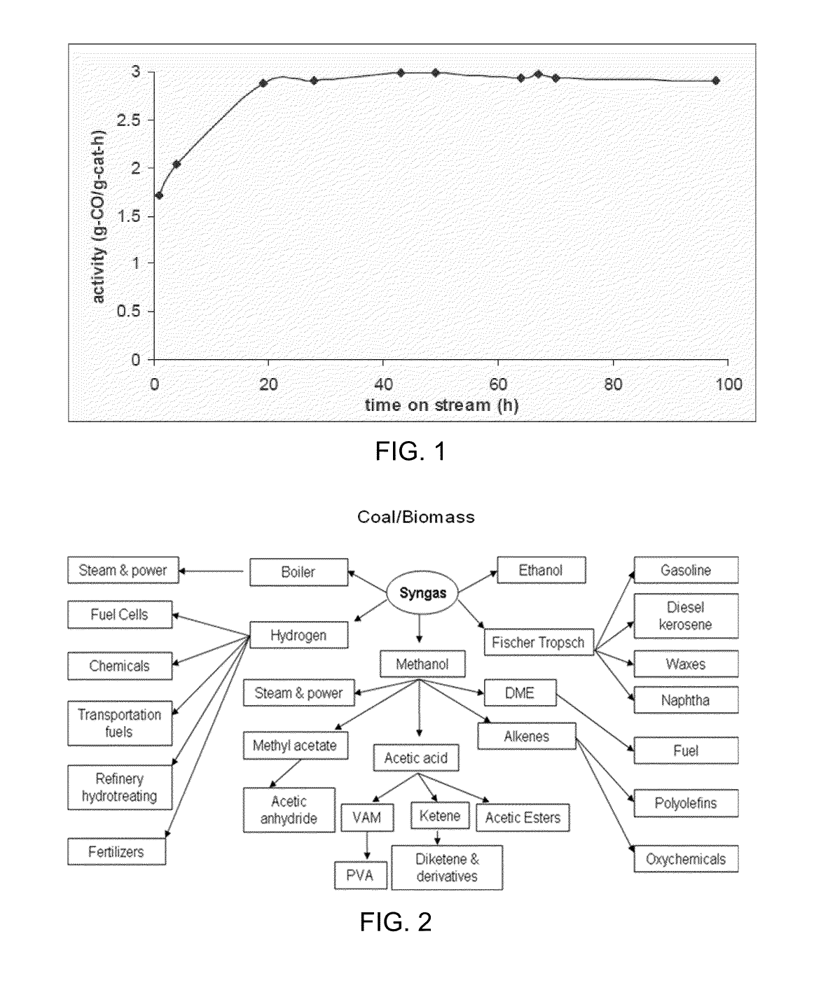

The catalyst was evaluated for F-T activity in a laboratory scale fixed-bed reactor. The reactor was charged with 250 mg of the reduced catalyst (UT-1-1.5) mixed with 2.0 g of silicon carbide (SiC) diluent. The catalyst-SiC mixture was held in place with a Whatman QMA quartz fiber filter. The vertical reactor tube was heated by a cylindrical heating furnace with the reactor tube fixed into the middle of the cylindrical furnace. The furnace was also equipped with a thermocouple for temperature control. H₂, CO and Ar (an internal standard) were introduced into the reactor by means of three mass flow controllers (Omega FMA 5400/5500). The H₂:CO ratio was maintained at 2:1. The reactant gases, H₂ and CO, were passed at a flow rate of 50 mL/min and 25 mL/min, respectively. The flow rate of Ar was maintained at 12.5 mL/min. The pressure inside the reactor tube was maintained at ˜100 psi with a back pressure regulator and the reactor tube was subsequently heated to 265° C. The catalyst was evaluated at 265° C. and a pressure of 100 psig for ˜100 hrs. The F-T products in the C₁-C₄ range were analyzed online with a gas chromatograph (GC) equipped with a silica gel column and TCD and FID detectors. A cryogenic trap maintained at −78° C. using a 2-propanol-dry ice mixture, an ice bath trap and a room temperature trap were placed before the GC to collect all the hydrocarbon fractions and water prior to the injection of the gaseous stream (C₁-C₄ fraction) into the GC. The composition of the heavier fraction collected in all the traps was analyzed by gas chromatography-mass spectroscopy (GC-MS) on a Hewlett Packard (5897 series) instrument equipped with a HP-DB5 column and a single quadrupole detector. It was observed that the UT-1-1.5 catalyst started showing high conversion early during the run. The catalyst functioned without much loss of activity during the 100 h run. The activity of UT-1-1.5 with time on stream is showing in FIG. 1.

Ferrihydrite Nanoflakes Characterization

Iron catalysts can be ideal for use with hydrogen-lean syngas produced from coal gasification (adjusting the ratio of H₂ to CO). Iron catalysts can be incorporated onto various supports such as metal loading onto supports (i.e. Aerogels/Xerogels). This can be accomplished by wet impregnation (e.g. support immersed in metal salt solution to dope the metal onto the support) or gas phase incorporation (GPI) (e.g. support exposed to volatile organometallic precursors). Supercritical drying of wet gels, aerogels (90% of which volume is air, high surface area). Air drying of wet gels, xerogels (collapsed structure with low surface area and pore volumes). Relevant material on formation of these supports can be found in U.S. patent application Ser. No. 11/725,168, filed Oct. 25, 2007 which is incorporated herein by reference.

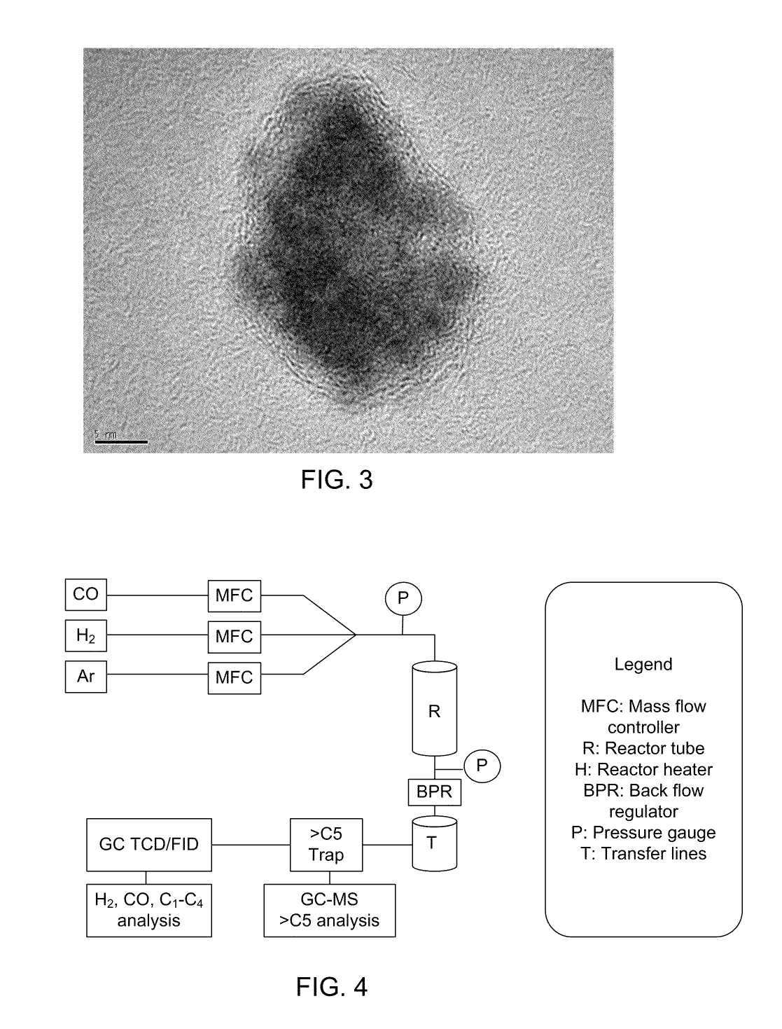

Ferrihydrite (FeOOH.nH₂O) nanoflakes can be formed. Novel structural pattern for the 2 Line-Ferryhydrite (2L-FHYD) nano-flakes, without any noticeable crystalline order but with an effective magnetic size of about 2.5 nm is show in the TEM image of FIG. 3. The unique structures and properties of the newly synthesized FHDY catalysts are apparently due to the presence of the added aluminum and copper. FHYD materials exhibited a very high BET surface area of ˜311 m²/g.

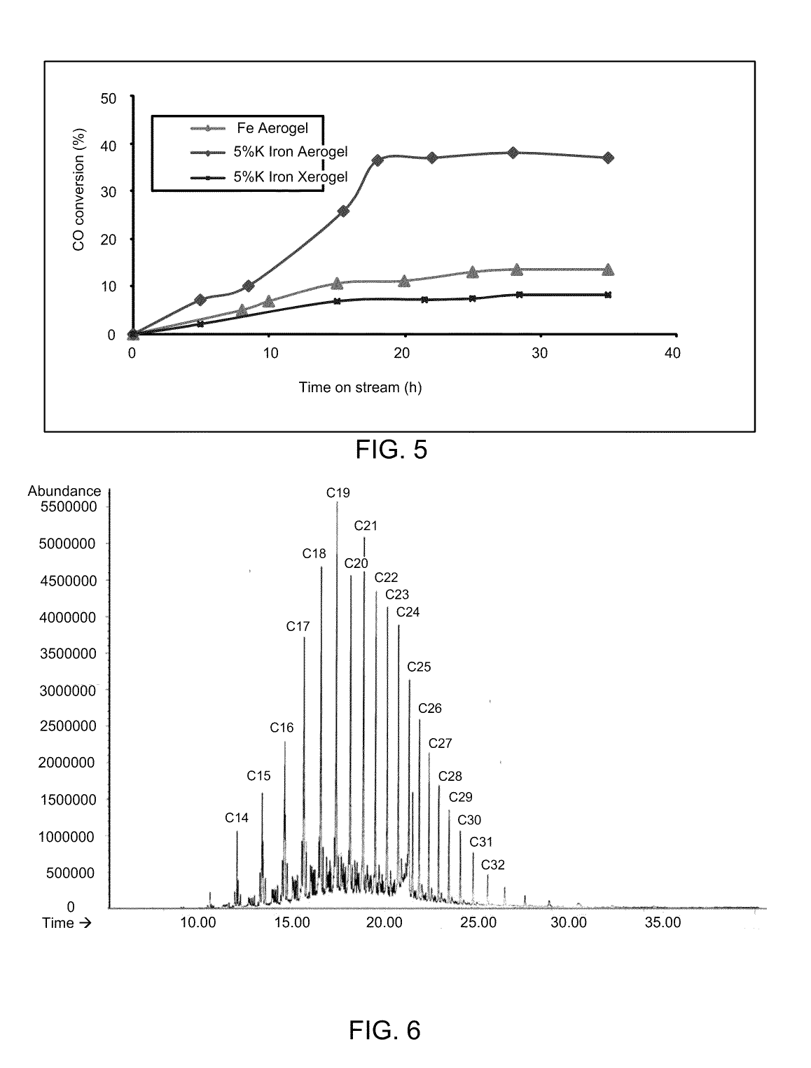

A fixed bed reactor system for F-T synthesis can be used as shown in FIG. 4. F-T activity of potassium promoted iron aerogel and xerogel catalyst in a fixed bed reactor was also evaluated. FIG. 5 shows catalyst evaluation in F-T reaction in a fixed bed reactor at 265° C., 100 psi. FIG. 6 is a XRD spectrum showing ˜80% of the fuel products were in the diesel range.

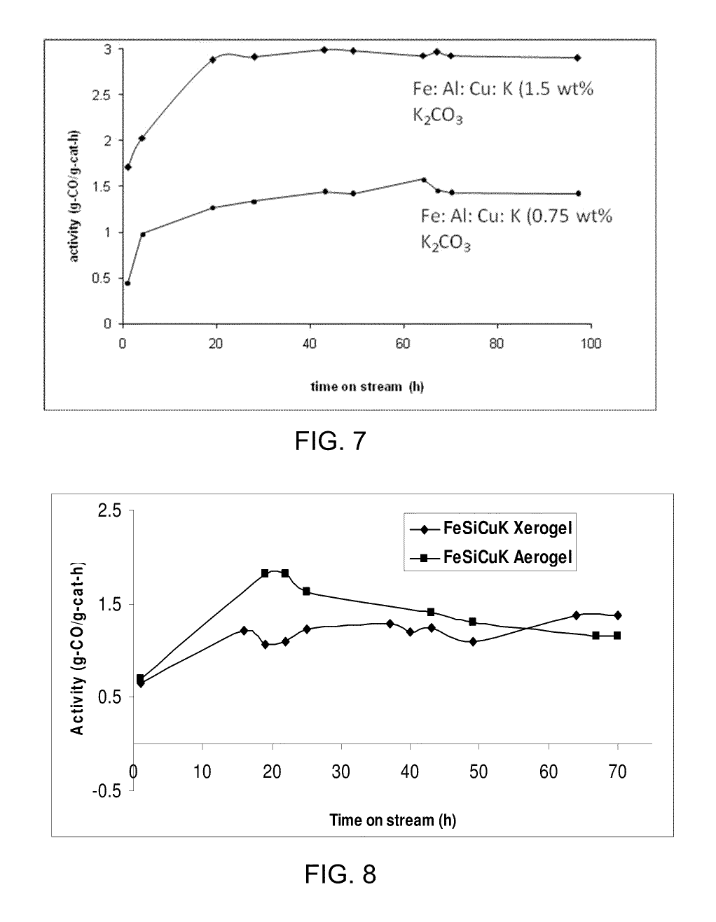

Activity was greater for the potassium promoter doped iron aerogel compared to undoped iron aerogel. Potassium incorporated iron aerogel catalyst shows a comparable conversion as 20 wt % Fe loaded onto high surface area SBA-15 support with ˜80% product in diesel range. Iron aerogel catalyst changed from a non-rigid open aerogel structure to an iron carbide/metallic iron agglomeration with no discernible loss of catalytic activity. F-T activity of ferrihydrite catalyst was evaluated in a fixed bed reactor CO:H₂ 1:2, 265° C., 100 psi. FIG. 7 illustrates the affect of variation in potassium content.

Ferrihydrite nanoflakes exhibited high F-T activity with no loss of activity in a ˜100 h run. F-T activity of ferrihydrite nanoflakes increases with promoter (potassium) content up to at least about 3 wt %.

F-T activity of mixed metal xerogels and aerogels catalyst was evaluated for Fe:Al:Cu (100:25:5) and Fe:Si:Cu (100:25:5). FIG. 8 illustrates that not much difference was achieved between aerogel/xerogel activity.

Characterization of the F-T products obtained with the synthesized catalysts can also be done using techniques such as carbon-13 NMR, gas chromatograph-mass spectrometry (GCMS), etc. Mixing and evaluation of F-T catalysts with zeolites (e.g. ZSM-5 etc) for cracking and aromatization of long-chain paraffins formed with the F-T catalysts to gasoline/diesel-range products for one pot synthesis to produce commercial F-T fuels was also shown by this example.

### Example 2

The synthesis, detailed characterization and use of Al and Cu doped amorphous nanoflakes of a 2-line ferrihydrite (FHYD) are described. The 2-line FHYD nanoflakes were characterized by XRD, TEM and Mössbauer spectroscopic techniques, etc., and used after modification with a promoter (K₂CO₃) for the production of liquid fuels from syngas. The synthetic ferrihydrite nanoflakes exhibited high conversion of syngas to liquid fuels in a fixed bed reactor for up to ˜100 h without loss of activity. A detailed characterization of the synthetic Al and Cu doped ferrihydrite nanoflakes indicates that the structural and magnetic properties of these ferrihydrite nanoflakes are substantially different from conventional 2L-FHYD.

### Synthesis of Fe:Al:Cu Ferrhydrite (UT-1) (Fe:Al:Cu 100:25:5 by wt)

Starting materials of Fe(NO₃)₃.6H₂O, Al(NO₃)₃.9H₂O and Cu(NO₃)₂.2.5H₂O and Na₂CO₃ were used as received (Aldrich). The hydrated nitrates of Fe(III) (36.18 g, 89.60 mmol), Al(III) (17.36 g 46.30 mmol), and Cu(II) (0.9188 g, 3.960 mmol) were dissolved in 200 mL ultrapure water (Mili-Q). The metal salt solution (containing Fe:Cu:Al) and a separate Na₂CO₃ solution (22.02 g, 207.7 mmol in 200 mL ultrapure water) were then added drop wise simultaneously through burettes into a beaker with vigorous stirring. An orange colored precipitate started forming. After complete addition of both of the solutions, the precipitate was aged (with stirring) for another 24 hrs at room temperature. The orange colored precipitate (UT-1) was filtered and washed with a large quantity of deionized water.

Impregnation of Fe:Al:Cu Ferrhydrite with Potassium Carbonate (F-T Promoter).

The ferrihydrite precipitate (UT-1) was dried in air in an oven at 100° C. overnight and impregnated with 1.5 weight % K₂CO₃ using the previously described wet impregnation technique to obtain a Fe:Al:Cu:K ferrihydrite catalyst.

Calcination of Fe:Al:Cu:K Ferrihydrite.

Calcination was carried out by heating the Fe:Al:Cu:K ferrihydrite in a glass dish in a programmable furnace which was ramped at 10° C./min up to 400° C. in air flowing at 200 mL/min. The temperature of the furnace was held at the predetermined temperature of calcination (400° C.) for 4 hours and subsequently cooled slowly to ambient temperature to get UT-2 ferrihydrite.

Reduction of Fe:Al:Cu:K Ferrihydrite.

The UT-2 catalyst was reduced prior to testing for the Fischer-Tropsch reaction under pure hydrogen (flow rate 50 mL/min) by heating the UT-2 in a glass dish in a programmable furnace which was ramped at 10° C./min up to 280° C. The temperature of the furnace was held at the temperature of reduction (280° C.) for 8 hours and subsequently cooled slowly to ambient temperature to get UT-3 (catalyst F-T synthesis).

Surface Area Measurements.

To evaluate the surface area of the ferrhydrite catalysts, nitrogen adsorption isotherms were determined using a Micromeritics Chemisorb instrument (Model 2720). Isotherms were used to calculate the BET specific surface areas. Measurements were carried out under a nitrogen flow rate of 12 mL/min for all the samples. The samples were pretreated before the BET surface area measurements by degassing for 40 mins under nitrogen flowing at a rate of 12 mL/min at 120° C.

X-ray Diffraction, Electron Magnetic Resonance (EMR) Spectroscopy.

The phases of metals present in the UT-1, UT-2, reduced UT-3 form and spent catalyst UT-11 (after F-T run) samples were determined by X-ray diffraction and electron magnetic resonance spectroscopy. Cu XANES spectra for samples UT-1, UT-2 were recorded at beam-line X-18B of the National Synchrotron Light Source (NSLS), Brookhaven National Laboratory, NY. The TEM images were recorded at the University of Kentucky.

Iron Mössbauer Spectroscopy

The phases of iron present in the prepared ferrihydrite nanoflakes catalyst both before and after (spent catalysts) being used in the F-T synthesis were determined by iron Mössbauer spectroscopy. The Mössbauer spectra were obtained at room temperature using a conventional constant-acceleration spectrometer. The spectrometer consisted of a Halder, GmbH, Mössbauer drive and control unit interfaced to a personal computer by means of PHA/MCS boards from Can berra Nuclear. The Mössbauer spectra were collected in symmetric mirror-image mode over 1024 channels and the data were then folded to provide a 512-channel spectrum with an enhanced signal/noise ratio. A spectrum of metallic iron in thin foil form was collected at the opposite end of the Mössbauer drive simultaneously to the data collection for the unknown sample. This spectrum served to calibrate the velocity (energy) scale of the spectrum and to provide the means to establish the folding point of the spectrum. The transmission of the 14.4 keV Mössbauer gamma rays through the sample and calibration foil was measured by means of gas-filled proportional counters. The data collection was synchronized to the velocity of the oscillating source by means of a start pulse from the Halder control unit and utilized a dwell time of 100 μs/channel. The velocity range for the data accumulation was set to ±4 mm/s for the three original samples and to ±12 mm/s for the reacted sample due to the presence of magnetically split components in its spectrum.

Analysis of the Mössbauer spectra of these samples consisted of fitting the data to combinations of two-peak quadrupole components for the original unreacted samples and to six-peak magnetic hyperfine components for the reacted sample. A Lorentzian shape was assumed for the peaks in both the quadrupole and magnetic components. A model function was initially calculated based on the observed number of individual components in the spectrum and a least-squares fitting routine refined the model until convergence (closest agreement between model and data) was achieved based on minimization of the statistic, χ². Mössbauer parameters such as the isomer shift (IS, mm/s relative to metallic Fe), quadrupole splitting (QS, mm/s) and the magnetic hyperfine splitting, (H0, kGauss) were calculated for each component based on the fitted positions of the peaks in the component. Line widths and the areas under each component were also determined in the least-squares fitting. The percentages of iron determined for each component are based on the areas underneath the individual components relative to the total absorption by the iron in the sample.

Catalyst Evaluation for F-T Activity.

The synthetic ferrihydrite was evaluated after pretreatment (calcination followed by reduction) for F-T activity in a laboratory scale fixed-bed reactor system as depicted in FIG. 4. The reactor was charged with 250 mg of the fresh catalyst (sieved 30-45) mixed with silicon carbide (diluent) that was held in place with a Whatman QMA quartz fiber filter. The vertical reactor tube was heated by a cylindrical heating furnace with the reactor tube fixed into the middle of the cylindrical furnace. The furnace was also equipped with a thermocouple for temperature control. H₂, CO and Ar (an internal standard) were introduced into the reactor by means of three mass flow controllers (Omega FMA 5400/5500). The H₂:CO ratio was maintained at 2:1. The reactant gases, H₂ and CO, were passed at flow rates of 50 mL/min and 25 mL/min, respectively along with argon as internal standard at a flow rate of 12.5 mL/min. The pressure inside the reactor tube was maintained at ˜100 psi with a back pressure regulator (TESCOM Corp. Model ER 3000 Sl-1). The F-T products in the C₁-C₄ range were analyzed online with a gas chromatograph (SRI 8610 C) equipped with a silica gel column and TCD and FID detectors. An air trap (at ambient temperature), ice trap (0° C.) and a cryogenic trap maintained at −78° C. using a acetone-dry ice mixture were placed before the GC to collect heavy hydrocarbon fractions and water prior to the injection of the gaseous stream into the GC. The composition of the heavier fraction collected in the traps preceding the gas chromatograph was analyzed by quantitative carbon-13 nuclear magnetic resonance (NMR) and DEPT NMR spectra for detailed characterization of the F-T fuel formed during the reactor run.

Characterization of F-T Oil and Wax by Quantitative Carbon-13 and DEPT NMR

The hydrocarbon type distribution and the relative amount of each carbon type (i.e. proton multiplicity of each carbon and quantitating the various carbon types) present in the F-T oils were investigated using Quantitative ¹³C NMR (Nuclear magnetic resonance) and the DEPT experiments. The experiments were performed on 500 MHz Varian Instrument (125.64 MHz for ¹³C resonance frequency, Pfg5sw probe). The samples were prepared in deuterated chloroform using 5 mm sample tubes. Tetramethylsilane (TMS) was used as an internal standard. Chemical shifts of all the carbon signals were calibrated with respect to TMS. CDCl₃ solvent gives a triplet located at 77.23 ppm in the carbon-13 spectrum. All single pulse spectra are obtained under quantitative conditions using a 45 degree pulse, and a pulse delay time of 45 s, which is five times the longest carbon spin-lattice relaxation time (T1), to ensure complete relaxation of the sample, with 2000 scans to ensure good signal-to-noise ratios, and with inverse gated decoupling.

Results and Discussion

Quantification of the elements present in synthesized ferrihydrite naoflakes (UT-1) was done by elemental analysis. The elemental analysis was performed (at Enviropro Laboratories, Salt Lake City) using acid digestion followed by analysis using the inductively coupled plasma atomic emission spectroscopy (ICP-AES) technique with a Perkin-Elmer Optima 3000 DV instrument. The ratio of (Fe:Al:Cu) was found to be 106:25:5 by weight. The synthesized ultra small Fe:Al:Cu ferrihydrite (UT-1) exhibited a very high surface area of 311 m²/g. After impregnation with potassium carbonate and subsequent calcination at 400° C., the ferrihydrite UT-2 lost some but still exhibited a reasonably high surface area of 230 m²/g.

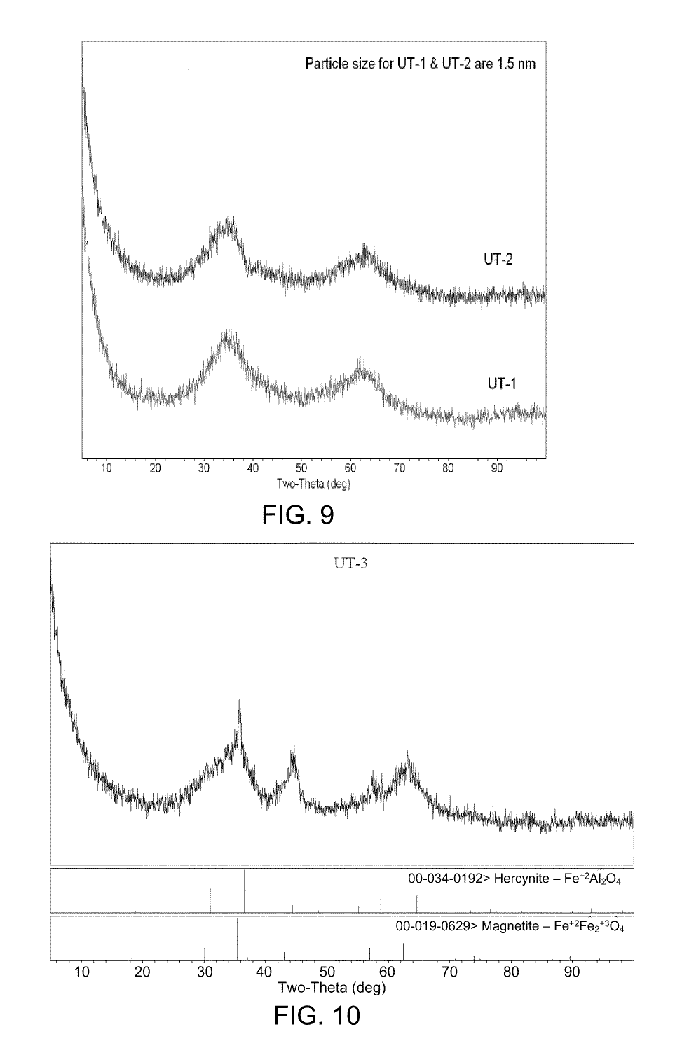

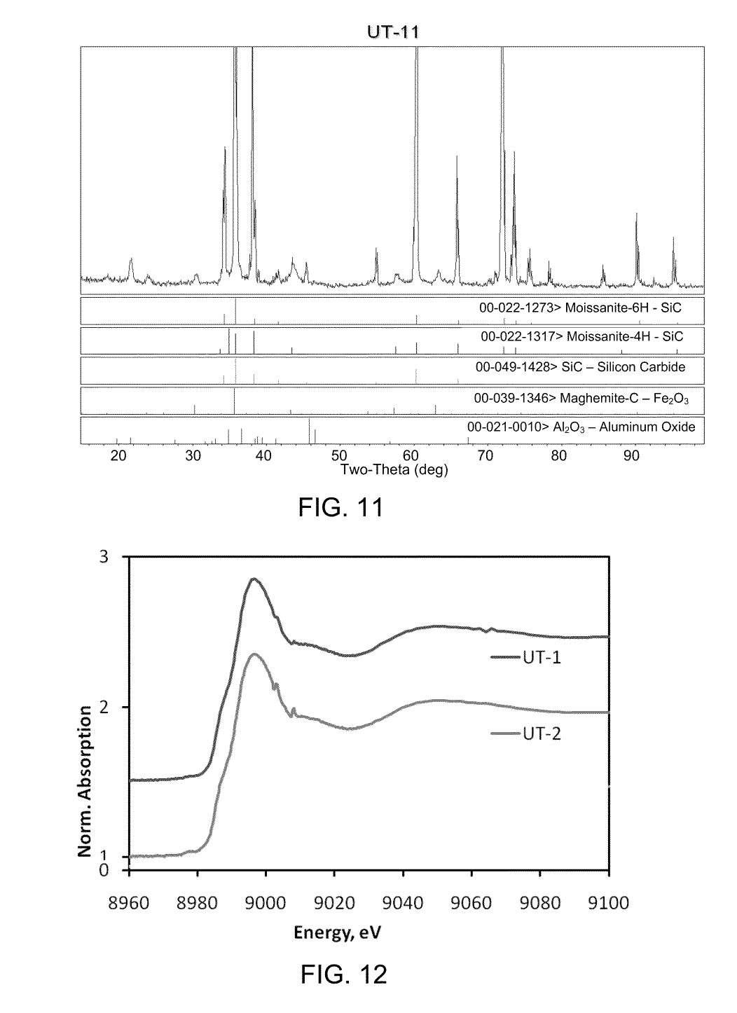

Detailed characterizations of various phases of metals present in the as prepared Fe:Al:Cu ferrihydrite (UT-1) and after impregnation with potassium carbonate followed by calcination, Fe:Al:Cu:K ferrihydrite (UT-2), were carried out by X-ray diffraction, electron magnetic resonance spectroscopy (EMR), iron Mössbauer spectroscopy and electron microscopy. The XRD patterns of UT-1 and UT-2 are shown in FIG. 9. For UT-1 and UT-2, there was essentially no difference in the XRD spectra since both showed the broad lines expected from the ferrihydrite structure with a particle size of about 1.5 nm. For an F-T run, the catalyst UT-2 was pretreated (reduced under pure H₂ as previously described) and then loaded in the fixed bed reactor. The reduced catalyst (UT-3) also had a structure of 2-line ferrihydrite (FHYD) as shown in FIG. 10. In addition, some of the FHYD was converted to FeAl₂O₄, Fe₃O₄ or both. Thus, the original two-line ferrihydrite structure (FeOOH.nH₂O) of UT-2 was still present in reduced UT-3 but there were some additional lines due to magnetite and FeAl₂O₄ which apparently result from the partial reduction of ferrihydrite. Clearly, reduction of ferrihydrite to Fe is not complete. The XRD pattern for the spent UT-11 catalyst (after the F-T reaction run) was also recorded and is shown in FIG. 11.

The XRD pattern of the spent UT-11 after the Fischer-Tropsch reaction matches with three forms of SiC, maghemite and possibly aluminum oxide. Due to the low concentration of Cu and K (Fe:Cu:Al:K ratio of 100:5:25:1.5) it was difficult to detect the presence of Cu/K compounds. Evidently, FHYD was converted to maghemite/magnetite after the F-T run. The Cu-XANES spectra for UT-1 and UT-2 samples were also recorded and are shown in FIG. 12. The Cu-XAFS spectra are rather weak and noisy due to the presence of 20 fold greater quantities of Fe in their formulations which contributes strongly to the fluorescent background and hence could not be recorded satisfactorily. As a result, only the Cu XANES spectra provided useful information. The Cu XANES spectra of UT-1 and UT-2 shown in FIG. 12, are quite similar and show that the oxidation state of the copper in these catalyst formulations is Cu²⁺.

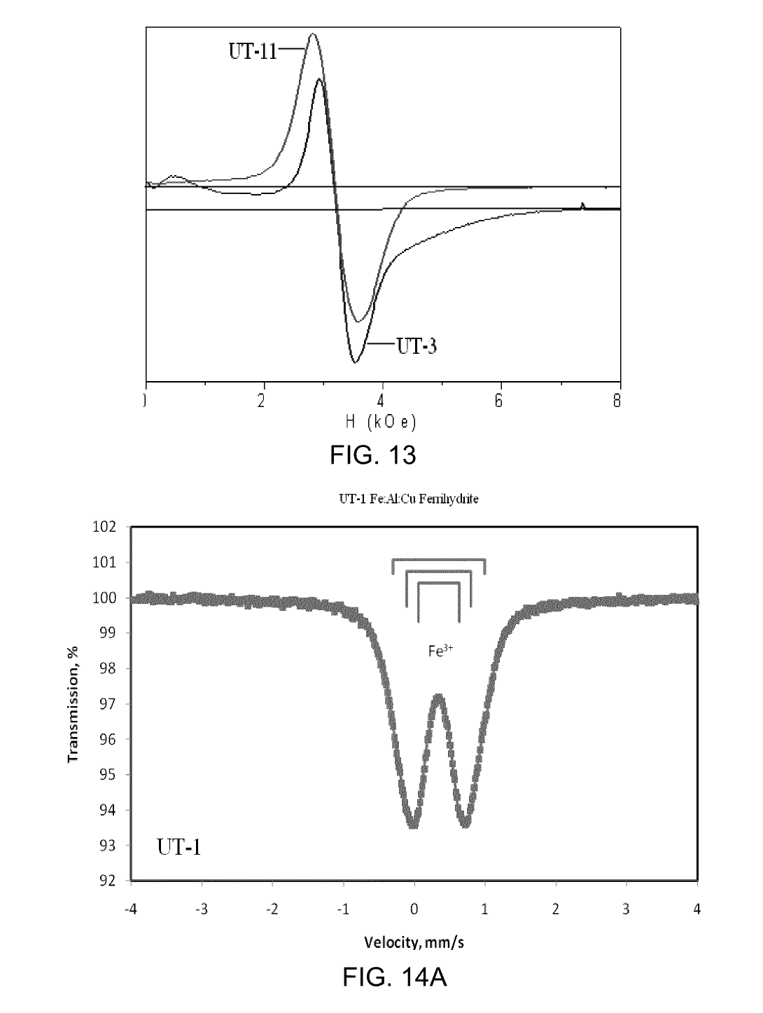

The EMR spectra for the F-T catalyst UT-3 both before and after the F-T run were recorded and are shown in FIG. 13. The g values for the UT-3 and spent catalyst sample UT-11 were 2.056 and 2.092, respectively. Both UT-3 and the spent catalyst UT-11 showed strong EMR spectroscopy absorption signals. These signals are centered near the free electron value of g≈2 with an additional broad absorption for UT-3. Both Fe₃O₄ and γ-Fe₂O₃ yield EMR signals near g=2, confirming the presence of Fe₃O₄ in UT-3 and γ-Fe₂O₃ in the spent UT-11 catalyst after the F-T reaction run, as indicated by X-ray diffreaction. The broader resonance in UT-3 may be due to FeAl₂O₄ as indicated by XRD measurements.

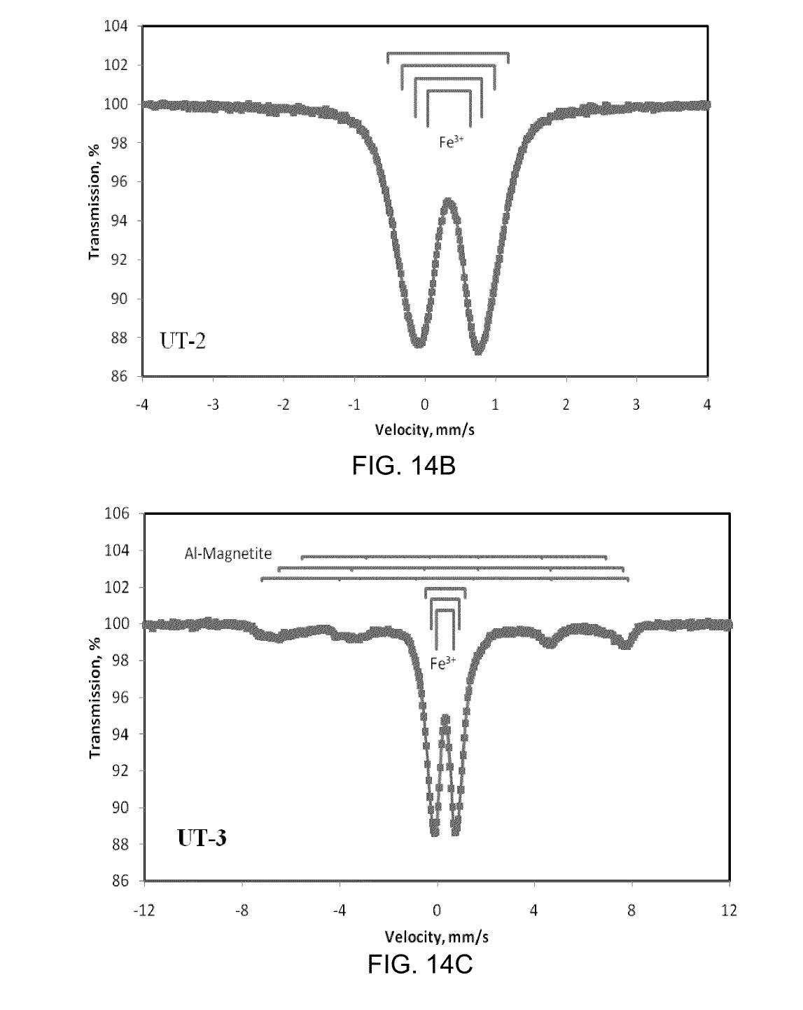

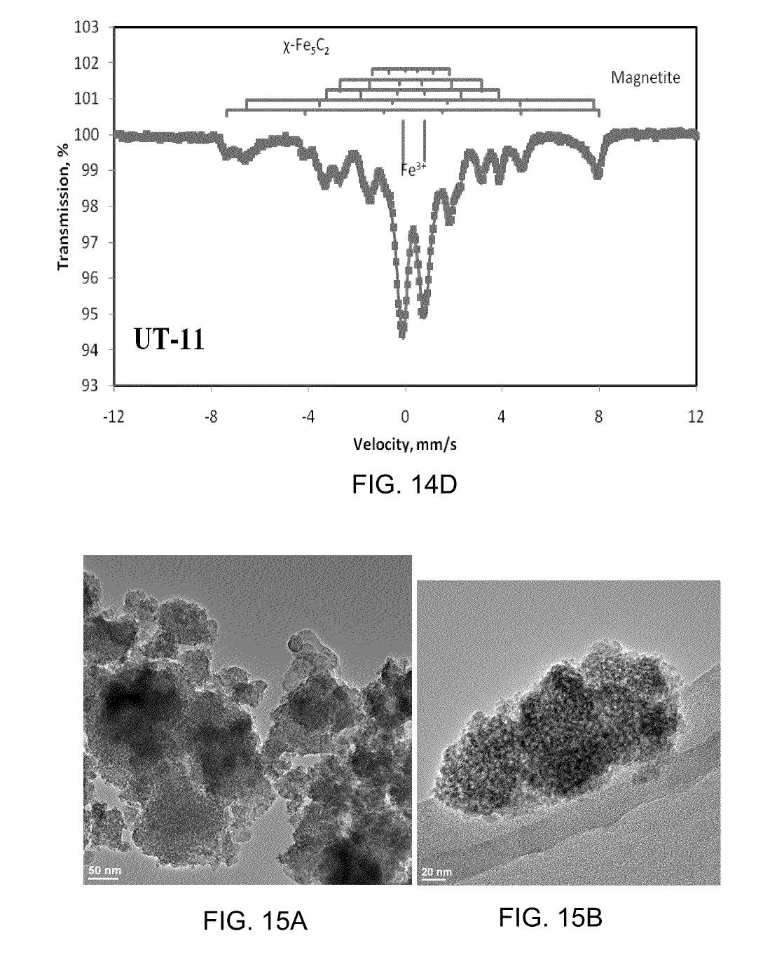

To further corroborate the XRD data and determine the phases of iron present in the catalyst, Mössbauer spectroscopy was carried out on UT-1, UT-2, UT-3 as well as spent catalyst (UT-11) as shown in FIGS. 14A-14D. Results indicate that the Fe is present entirely as ferrihydrite in UT-1 and UT-2. The reduced UT-3 catalyst has iron predominantly as Fe³⁺ with small amounts of FeAl₂O₄, Fe₃O₄ or both. The spent catalyst (after F-T reaction) UT-11 consists of non-magnetic Feⁿ (either unreacted catalyst precursor or very small particle ferric oxide), non-stoichiometric magnetite, and the Hagg Carbide, Fe₅C₂. The approximate proportion of iron present was found to be 41% in the ferric oxide, 23% in the form of magnetite and 36% as carbide. Non-stoichiometry in magnetite is likely due to substitution of Al or other cations for Fe in the magnetite structure. This also accounts for the broadness of the B-site absorption. The transmission electron microscopy (TEM), on ferrhydrite samples UT-1 and UT-2 (FIGS. 15A and 15B) showed nanoflakes of about 5-20 nm size consisting of clusters of atoms.

No diffraction fringes were observed in the TEM images, thus indicating that the synthesized ferrihydrite was essentially amorphous. The clustering of nanoflakes signifies non-uniformity of thickness of the nano-flakes. The observed flake-like morphology and the amorphous nature of this sample likely result from Al and Cu doping. Further detailed magnetic characterization and properties of these ferrihydrite nanoflakes were also made. The magnetic properties of theses nanoflakes were found to be substantially different from conventional 2L-ferrihydrite.

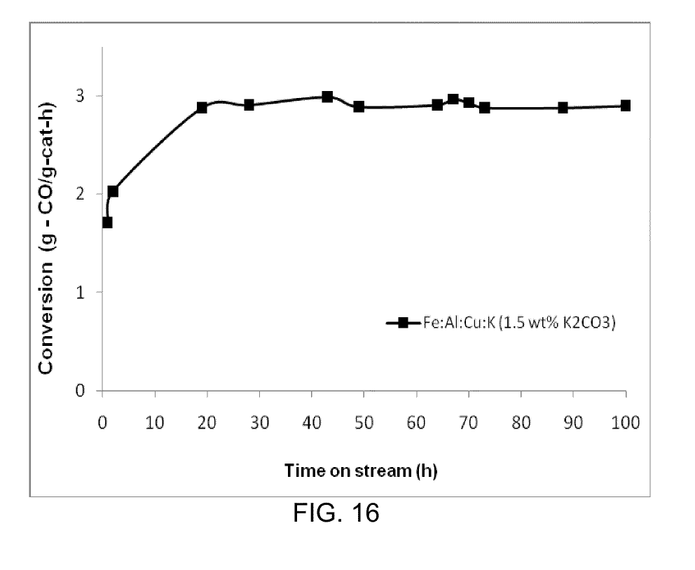

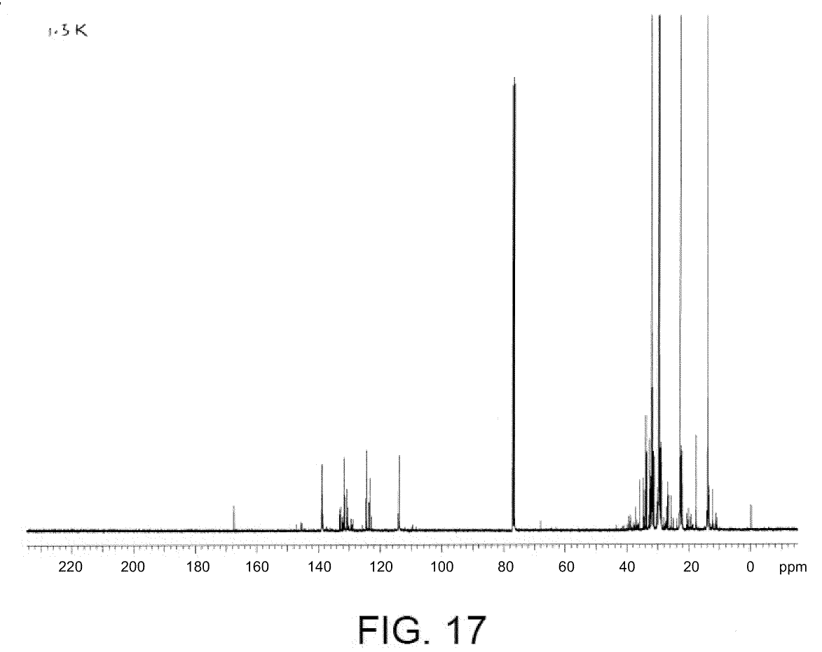

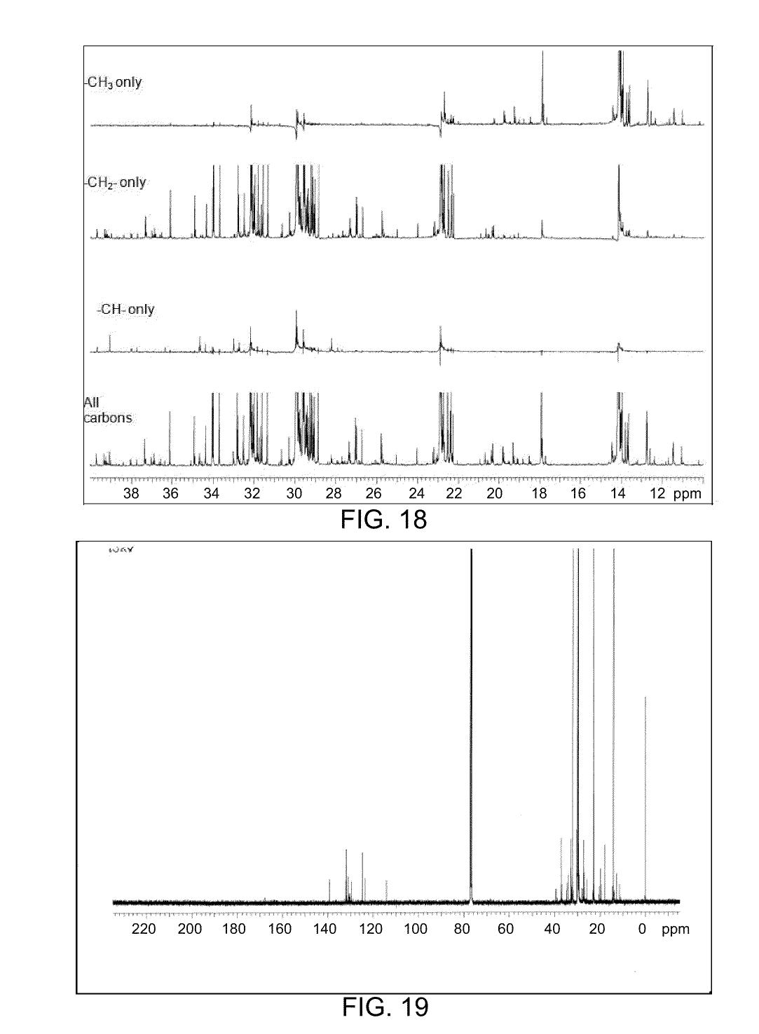

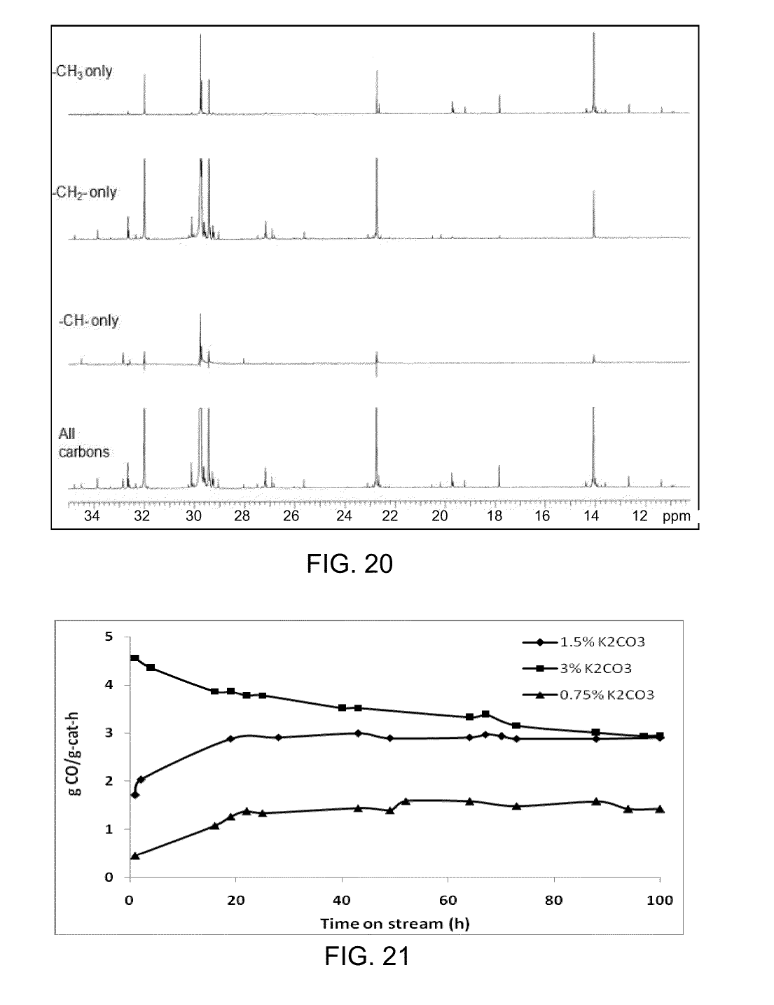

The ferrihydrite nanoflakes (after pretreatment i.e. calcination and reduction) were evaluated for F-T activity in a fixed bed reactor at a pressure of 100 psi and a temperature of 265° C. The catalyst was tested for ˜100 h for F-T activity. The catalyst was found to have excellent F-T activity. Specifically, the CO conversion in terms of g-CO/g-cat-h versus the time on stream is shown in FIG. 16. The catalyst functioned without much loss of activity during the 100 h run. A heavy product fraction in the form of wax was collected in the trap maintained at room temperature and the remaining lighter fractions (oil) were collected in cryogenic traps maintained at 0° C. (using ice) and −78° C. (using acetone: dry ice mixture). The wax obtained in the heavy fraction trap was white in color. The colorless F-T oil obtained was dried using drierite. Both the F-T wax as well as oil collected were subjected to detailed characterization by using quantitative carbon-13 NMR spectroscopy and distortion less enhancement by polarization transfer (DEPT) experiments. The detailed quantitative carbon-13 NMR and DEPT NMR for the F-T oil are shown in FIGS. 17 and 18, respectively, while the quantitative carbon-13 NMR and DEPT NMR for the F-T wax obtained with the ferrihydrite catalyst are shown in FIGS. 19 and 20, respectively.

Carbon-13 NMR spectrometry was used to detect the carbon types directly yielding total aliphatic carbon (Cₐₗ), total paraffinic carbon (Cₚ) and total olefinic carbon(Cₒₗ) in the samples (F-T oil as well as wax). The DEPT experiments generated sub-spectra of different CHₙ, (n=1-3) groups. These sub-spectra were interpreted to estimate average structural parameters of the F-T product. The percentages of various carbon types were calculated as follows: a) Total aliphatic carbon(Cₐₗ): ratio of sum of integrated intensity of all signals in the aliphatic region (10-45 ppm) to the sum of total integrated intensity (10-180 ppm) excluding solvent and TMS. b) Total paraffinic carbon(Cₚ): ratio of the sum of integrated intensity of sharp and well resolved resonances characteristic of n-parrafin (10-45 ppm) (Ip) to the total integrated area excluding solvent and TMS. c) Total olefinic carbon (Cₒₗ): ratio of the sum of integrated intensity from 110-140 ppm to the total integrated area excluding solvent and TMS. The chemical shifts observed in the F-T wax spectrum at 32.8, 30.6, 30.5, 30.2, 23.5, 14.7 ppm are characteristic of long chain normal paraffin. A CH₂ resonance around 30.0 ppm corresponds to large amounts of methylene carbons present in very long alkyl chains in the F-T wax as well as F-T oil sample. Average chain length of n-paraffins (CL) was calculated as (CL)=2 I_{P}/I_{(t-CH)} where I_{P} is the sum of integrated intensity of sharp and well resolved resonances characteristic of n-parrafin (10-45 ppm), and I_{(t-CH)} is the integral intensity of terminal CH₃ at 14.0 (sharp). The average chain lengths for the F-T oil and wax were found to be 10 and 19, respectively. In the DEPT spectrum of F-T oil, the chemical shifts for —CH₃ (17.8, 19.3 ppm) and —CH (33.0, 39.0 ppm) are characteristic of methyl branching along the paraffin chain. The details of various carbon types and parameters calculated for the F-T oil and wax are given in Table 1.

TABLE 1

| Types of carbon | F-T Oil | F-T wax |
|---|---|---|
| Total aliphatic carbon*(Cₐₗ) | 88.0 | 96.8 |
| Total paraffinic carbon* | 77.4 | 86.0 |
| (Cₚ) | | |
| Total olefinic carbon*(Cₒ) | 12.0 | 3.0 |
| Aliphatic methine | 8.3 | 7.4 |
| carbon**, CH | | |
| Aliphatic methylene | 68.7 | 73.8 |
| carbon**, CH₂ | | |
| Aliphatic methyl carbon**, | 23.1 | 18.8 |
| CH₃ | | |
| Average Chain length* | 10.0 | 19.1 |

*calculated from quantitative carbon-13 NMR
**calculated from DEPT experiment

The total aliphatic carbon content was 88% and 96.8% for the F-T oil and F-T wax, respectively. In all, the data show that F-T oil obtained is constituted mainly of straight chain n-paraffins with an average chain length ˜10 which is in the range for the middle distillate (kerosene/Jet fuel) range fuel(C₆-C₁₂). The olefinic content in the F-T oil was ˜12%.

The unique morphology of this sample combined with its smaller effective volume resulted in excellent catalytic properties as corroborated by the ˜100 h F-T run in a fixed bed reactor for the conversion of syngas to Fischer-Tropsch (F-T) fuels. The catalyst showed high CO conversion and stability over the entire F-T run. Both F-T oil and wax were obtained as products. The F-T wax was a characteristic carbo-wax as corroborated by the NMR studies. The F-T oil obtained was constituted mainly of normal paraffins with an average carbon chain length of 10 lying in the carbons/molecule range for a middle distillate fuel (jet fuel/kerosene). This synthesized ferrhydrite nanoflake catalyst can also be modified by mixing with an acid catalyst (e.g. ZSM-5, etc.,) to shift product distribution toward the formation of high octane gasoline/middle distillate range isoparaffins and aromatics. The target is the transformation of the primary F-T products (heavier hydrocarbons and waxes) on the zeolite acid sites by cracking of heavier hydrocarbons, skeletal isomerization, hydrogen transfer, and aromatization of the short-chain olefins.

### Example 3

### Variation of Activity of FHYD (Fe:Al:Cu 100:25:5 by Weight) Catalyst with Variation in Promoter (K₂CO₃) Content

The Fe:Al:Cu FHYD nanoflakes were impregnated with 0.75 wt %, 1.5 wt % and 3 wt % of K₂CO₃ promoter to study the effect of promoter content. After impregnation of the promoter (K₂CO₃), the FHYD catalyst was calcined (400° C., 10° C./min, 4 h soak time, as previously described in Examples 1 and 2). The calcined product was then reduced (280° C. at 10° C./min, soak time 8 h as in Examples 1 and 2) and finally loaded into the same fixed bed reactor from Example 1 and 2 for evaluation of F-T activity.

The activity vs time for 0.75, 1.5 and 3 wt % K₂CO₃ impregnated FHYD nanoflakes (Fe:Al:Cu 100:25:5 by wt.) catalyst is shown in FIG. 21.

Synthesis of Fe:Al:Cu Ferrhydrite (Fe:Al:Cu 100:25:10 by wt)

Hydrated nitrates of Fe(III) (36.17 g, 89.60 mmol), Al(III) (17.37 g 46.30 mmol), Cu(II) (1.831 g, 7.876 mmol) were dissolved in 200 mL ultrapure water (Mili-Q). The metal salt solution (containing Fe:Cu:Al) and Na₂CO₃ solution (22.4295 g, 207.7 mmol in 200 mL ultrapure water) were then added drop wise simultaneously through burettes into a beaker with vigorous stirring. An orange colored precipitate started forming. After complete addition of both of the solutions, the precipitate was aged (with stirring) for another 24 hrs at room temperature. The orange colored precipitate was filtered and washed with a large quantity of deionized water.

Impregnation of Fe:Al:Cu (100:25:10 by wt.) Ferrhydrite with Potassium Carbonate (1.5% wt).

The ferrihydrite precipitate was dried in air in an oven at 100° C. overnight and impregnated with 1.5 weight % K₂CO₃ using the wet impregnation technique to get the Fe:Al:Cu:K ferrihydrite

Calcination of Fe:Al:Cu:K (100:25:10:1.5) Ferrihydrite

The calcination was carried out by heating Fe:Al:Cu:K ferrihydrite in a glass dish in a programmable furnace which was ramped at 10° C./min up to 400° C. in air flowing at 200 mL/min. The temperature of the furnace was held at the predetermined temperature of calcination (400° C.) for 4 hours and subsequently cooled slowly to ambient temperature to get UT-2 ferrihydrite

Reduction of Fe:Al:Cu:K Ferrihydrite.

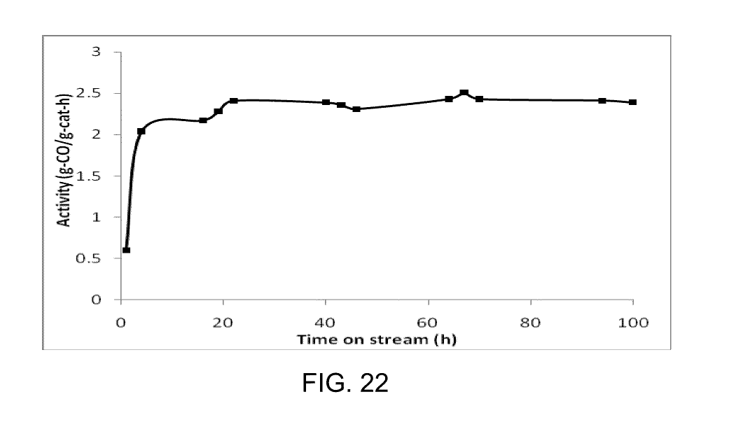

The calcined catalyst was reduced prior to testing for the Fischer-Tropsch reaction under pure hydrogen (flow rate 50 mL/min) by heating the UT-2 in a glass dish in a programmable furnace which was ramped at 10° C./min up to 280° C. The temperature of the furnace was held at the temperature of reduction (280° C.) for 8 hours and subsequently cooled slowly to ambient temperature to give F-T catalyst. The activity vs time for FHDY catalyst (Fe:Al:Cu:K 100:25:10:1.5) is shown in FIG. 22.

The exemplified compositions provide a synthesis of ultra small synthetic ferrihydrite (FHYD with D≈2.5 nm) which represents a new way to produce FHYD nanoparticles with ultra small dimensions not usually found in nature. High surface area of these nanoparticles makes them effective as catalyst precursors for the Fischer-Tropsch Synthesis of liquid fuels from syngas and other processes. These ultra small particle size (e.g. 2.5 nm) have high surface area ideal for catalysis applications. Despite the presence of other metals such as Al and Cu (usually absent in natural FHYD), a 2-line FHYD structure is obtained as confirmed by XRD and FTIR spectroscopy. The synthesis is a simple method and the resulting ultra small synthetic ferrihydrite exhibits high catalytic activity in the Fischer-Tropsch synthesis.

These materials can be used in production of alternative liquid fuels from syn gas that can be derived from coal/biomass. Also, the FHYD interact with environmentally important species such as arsenic, lead, mercury, phosphate and many organic species and can be used for their removal. For example, the FHYD compositions can be used in filters or otherwise contacted with contaminated fluids.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A ferrihydrite composition, comprising an ferrihydrite including a structural promoter metal, a chemical promoter metal and potassium to form an amorphous nanoparticulate.

2. The composition of claim 1, wherein the structural promoter metal includes at least one of Al and Si.

3. The composition of claim 2, wherein the structural promoter metal is aluminum.

4. The composition of claim 1, wherein the chemical promoter metal includes at least one of Cu, Mn, Pd, Ru, Cr, Pt, La, and Zn.

5. The composition of claim 4, wherein the chemical promoter metal is Cu.

6. The composition of claim 1, wherein composition has a X:Y:Z ratio where X is the weight of Fe, Y is the weight of structural promoter metal and Z is the weight of chemical promoter metal, wherein X is 100, Y is 20 to 30 and Z is 2 to 10.

7. The composition of claim 1, wherein the structural promoter metal is Al, the chemical promoter metal is Cu, and the composition has a Fe:Al:Cu ratio of about 100:25:5 by weight.

8. The composition of claim 1, wherein the potassium is present at about 0.4 to about 1.7 weight percent of the composition.

9. The composition of claim 1, wherein the nanoparticulate has an average size of about 5 nm to about 20 nm.

10. The composition of claim 1, wherein the nanoparticulate has a BET surface area prior to potassium loading from about 310 m²/g to about 380 m²/g.

11. A method of forming a ferrihydrite catalyst, comprising:
a) dissolving an iron salt, a structural promoter metal salt and a chemical promoter metal salt in water to form an aqueous iron solution;
b) precipitating a ferrihydrite solid from the aqueous iron solution by addition of a precipitating agent under conditions such that the ferrihydrite solid is a nanoparticulate;
c) incorporating a potassium into the ferrihydrite solid to form a ferrihydrite catalyst precursor; and
d) calcining the ferrihydrite catalyst precursor to form the ferrihydrite catalyst.

12. The method of claim 11, wherein the iron salt, the structural promoter metal salt and the chemical promoter metal salt are at least one of nitrate and sulfate salts.

13. The method of claim 12, wherein the precipitating agent is a basic solution.

14. The method of claim 11, wherein the conditions include a low temperature from about 20° C. to about 35° C.

15. The method of claim 11, further comprising incorporating the ferrihydrite catalyst onto a support material.

16. The method of claim 15, wherein the support material is at least one of an aerogel and a xerogel.

17. The method of claim 15, wherein the incorporating is accomplished by wet impregnation, gas phase incorporation, supercritical drying, or air drying.

18. A method of converting a synthesis gas to a fuel product, comprising:
a) contacting a ferrihydrite catalyst with the synthesis gas under reaction conditions sufficient to form a fuel product mixture, said ferrihydrite catalyst including a structural promoter metal, a chemical promoter metal and potassium to form an amorphous nanoparticulate.

19. The method of claim 18, wherein the reaction conditions include a pressure from about 75 psi to about 150 psi.

20. The method of claim 18, wherein the reaction conditions include a temperature from about 200° C. to about 280° C.

21. The method of claim 18, further comprising simultaneously contacting the synthesis gas with a zeolite catalyst.

22. The method of claim 18, wherein the ferrihydrite catalyst can be maintained under the contacting for a reaction time on stream of about 70 hours to about 120 hours with less than 5% loss in CO conversion activity.

23. The method of claim 18, wherein the reaction conditions include a H₂ space velocity from about 1.068 hr⁻¹ to about 2.136 h⁻¹ and a CO space velocity from about 7.5 hr⁻¹ to about 15 hr⁻¹.

24. The method of claim 18, wherein the fuel product includes less than about 0.7 wt % oxygenates.

25. The method of claim 18, wherein the contacting occurs in a fixed bed reactor or a slurry reactor.
